(19) United States
(12) Patent Application Publication
(10) Pub. No.: US 2022/0354452 A1
(43) Pub. Date: Nov. 10, 2022

(54) USE OF ULTRAFAST ELASTICITY IMAGING FOR DETECTING PANCREATIC CANCERS

(71) Applicants: INSERM (INSTITUT NATIONAL DE LA SANTÉ ET DE LA RECHRCHE MÉDICAL), Paris (FR); UNIVERSITÉ PAUL SABATIER TOULOUSE III, Toulouse (FR)

(72) Inventors: Julie Guillermet-Guibert, Toulouse Cedex 1 (FR); Nicole Therville, Toulouse Cedex 1 (FR)

(21) Appl. No.: 17/624,048

(22) PCT Filed: Jul. 1, 2020
(86) PCT No.: PCT/EP2020/068528

§ 371 Date: Dec. 30, 2021

(30) Foreign Application Priority Data
Jul. 2, 2019 (EP) 19305899.7

(51) Int. Cl. A61B 8/08

(52) U.S. Cl. CPC A61B 8/085; A61B 8/485

(57) ABSTRACT

Rapid, easy and early pancreatic cancer diagnosis and therapeutic follow up continue to necessitate an increasing attention towards the development of effective treatment strategies for this lethal disease. The non-invasive quantitative assessment of pancreatic heterogeneity is limited. Here, the inventors report the development of a preclinical imaging protocol using ultrasonography and shear wave technology in an experimental in situ pancreatic cancer model to measure the evolution of pancreatic rigidity. The inventors evaluated the feasiblity of a live imaging protocol by assessing pancreas evolution with Aixplorer technology across 37 weeks. Protumorigenic mutations induced a significant decrease of the rigidity of pancreatic tissue before tumors developed in correlation with the detection of senescent marker p16-positive cells. Thus the promising results indicate the potential of the shear wave elastography to support individualization of diagnosis in this most aggressive disease. Thus, the present invention relates to methods for determining whether a subject has or is at risk of having a pancreatic cancer by using ultrafast elasticity imaging.

### FIELD OF THE INVENTION

The present invention is in the field of oncology.

### BACKGROUND OF THE INVENTION

Pancreatic ductal adenocarcinoma (PDAC) is a dismal disease without effective therapeutic option except surgery. This disease is the most lethal of the common cancers, and is projected by 2030 to be the second highest cause of death due to cancer. The European Union has the highest incidence of pancreatic cancer in the world; incidence is increasing in France, and throughout European latin countries (1, 2). One of the major challenges facing research scientist and the clinical community is the fact that pancreatic cancer is a very heterogeneous disease. In particular, development and evolution of tumors present a great inter-individual variation, as well as significant heterogeneity within the tumor of each individual as assessed by their molecular genetic and genomic characterization (3). Understanding this heterogeneity in a multi-scale integrated manner is vital to enable the newly developed targeted therapies to be efficient (4). Heterogeneity is not simply a function of the cancer cells themselves, but encompasses also the manner in which cancer cells interact with other cells of the tumor microenvironment, also called stroma. This, in turn, impacts the cancer response to treatment therapies, which is different in each individual. As a consequence, treatment choice and its efficiency evaluation should be individualized and tested as such in preclinical settings, to help clinicians predict the best therapeutic option in the shorter timeframe possible, and then to assess the variablity of response of the newly developed therapeutic strategies (3). However, easy and predictive methods to assess tumor tissular composition, therapeutic options and early treatment efficiency are lacking both in clinical and preclinical settings.

Ultrasound echography (US) is an inexpensive, non-invasive method of diagnostic or treatment evaluation that can be performed easily and repeatedly. However, US images do not quantitatively measure the changes of the physical characteristics of the tumors, possibly indicative of the changes of tumor physiopathology, neither measure the physical changes occuring in pancreatic parenchyma before the detection of the tumors, which could help to diagnose earlier (5).

Two dimensional shear wave elastography (2D SWE) as opposed to point shear wave elastography (point SWE) is a recent live ultrafast imaging method which allows a longitudinal follow-up of tissue rigidity in time and space (6-8). The propagation of shear waves in the tissue correlates to tissue elasticity and the wave velocity is proportional to tissue elasticity (6). The propagation velocity of transverse shear waves in human liver fibrotic tissue is higher than in healthy liver parenchyma (7, 9). Interestingly, this value is measured without applying a constraint on the tissue when imaging, hence it could be used as an indirect measurement of solid stress, the latter being mostly measured ex vivo on dissected tumors from xenografts (10, 11).

Solid stress and tissue stiffness affect tumour growth, invasion, metastasis and treatment. Currently, several elastography procedures exist and measure tissue stiffness (7, 12) (13-15). While used in preclinical models such as subcutaneously injected tumors (16), the longitudinal assessment of experimental in situ intra-abdominal tumors, and in particular of pancreatic tumors with ultra fast 2D-SWE technology is limited (17). Others recently performed such measurements ex vivo after organ dissection in a genetically engineered mouse (GEM) model of colon cancer (18). As opposed to point measurements by other technologies, the real-time imaging by 2D-SWE allows in theory the assessment of the stability of the measurement and quantification of an average value of rigidity in a large region of interest for higher reliability.

### SUMMARY OF THE INVENTION

As defined by the claims, the present invention relates to methods for determining whether a subject has or is at risk of having a pancreatic cancer by using ultrafast elasticity imaging.

### DETAILED DESCRIPTION OF THE INVENTION

Rapid, easy and early pancreatic cancer diagnosis and therapeutic follow up continue to necessitate an increasing attention towards the development of effective treatment strategies for this lethal disease. The non invasive quantitative assessment of pancreatic heterogeneity is limited. Here, the inventors report the development of a preclinical imaging protocol using ultrasonography and shear wave technology in an experimental in situ pancreatic cancer model to measure the evolution of pancreatic rigidity. In particular, intrapancreatic tumors were genetically induced by mutated Kras and p53 in KPC mice. The inventors evaluated the feasiblity of a live imaging protocol by assessing pancreas evolution with Aixplorer technology across 37 weeks. Lethality induced by in situ pancreatic cancer was heterogeneous in time. The developed method successfully detected tumor mass from 19 weeks onwards at minimal 0.029 cm³ size. Elastography measurements using shear wave methodology had a wide detection range from 0.9 kPa to 110 kPa. Protumorigenic mutations induced a significant decrease of the rigidity of pancreatic tissue before tumors developed in correlation with the detection of senescent marker p16-positive cells. An intratumoral increased rigidity was quantified and found surprisingly heterogeneous. Tumors also presented a huge inter-individual heterogeneity in their rigidity parameters; tumors with lowest rigidity at detection had the tendency to evolve more in their rigidity and heterogeneity, as well as in their volume. Increase in rigidity in tumors detected by ultrafast elasticity imaging coincided with detection of tumors by echography and with the detection of the inflammatory protumoral systemic condition by non invasive follow-up and of collagen fibers by post-processing tumoral IHC analysis. Thus the promising results indicate the potential of the shear wave elastography to support individualization of diagnosis in this most aggressive disease.

Thus the first object of the present invention relates to a method of determining whether a subject has or is at risk of having a pancreatic cancer comprising determining the rigidity of the pancreatic tissue of the subject wherein the determined rigidity indicates whether the subject has or is at risk of having a pancreatic cancer.

As used herein the term “pancreatic cancer” or “pancreas cancer” as used herein relates to cancer which is derived from pancreatic cells. In particular, pancreatic cancer included pancreatic adenocarcinoma (e.g., pancreatic ductal adenocarcinoma) as well as other tumors of the exocrine pancreas (e.g., serous cystadenomas), acinar cell cancers, and intraductal papillary mucinous neoplasms (IPMN).

As used herein, the term “risk” in the context of the present invention, relates to the probability that an event will occur over a specific time period and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion. “Risk evaluation,” or “evaluation of risk” in the context of the present invention encompasses making a prediction of the probability, odds, or likelihood that an event or disease state may occur, the rate of occurrence of the event or conversion from one disease state to another. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion, thus diagnosing and defining the risk spectrum of a category of subjects defined as being at risk of conversion. In the categorical scenario, the invention can be used to discriminate between normal and other subject cohorts at higher risk. In some embodiments, the present invention may be used so as to discriminate those at risk from normal.

In some embodiments, the subject can be male or female. In some embodiments, the subject can be one who exhibits one or more risk factors for pancreatic cancer (e.g. alcohol consumption or cigarette smoking) or a subject who does not exhibit risk factors, or a subject who is asymptomatic for pancreatic cancer (e.g. in case of a screening test). In some embodiments, the method of diagnosing described herein is applied to a subject who suffers from a pancreatitis. As used herein, the term “pancreatitis” has its general meaning in the art and refers to a variety of diseases in which the pancreas becomes inflamed. Pancreatitis is thus inflammation of the pancreas that progresses from acute (sudden onset; duration <6 months) to recurrent acute (>1 episode of acute pancreatitis) to chronic (duration >6 months). Chronic pancreatitis (CP) occurs most commonly after one or more episodes of acute pancreatitis and involves ongoing or recurrent inflammation of the pancreas, often leading to extensive scarring or fibrosis. CP causes progressive and irreversible damage to the pancreas and surrounding tissues. Calcification of pancreatic tissues is common and often diagnostic of CP. In over 70% of cases, CP is associated with excessive and prolonged alcohol consumption. While alcoholism is the most common cause of CP, other causes include metabolic disorders and, more rarely, genetic disposition (hereditary pancreatitis).

According to the present invention, the rigidity of the pancreatic tissue may be determined by any routine method well known in the art. Preferably, the rigidity of the pancreatic tissue is assessed by elastography. Different elastography techniques can be used to obtain the tissue elastography images. These include Shear Wave imaging on the Aixplorer ultrasound machine (Supersonic Imagine), acoustic radiation impulse force imaging, vibro-elastography (U.S. Pat. No. 7,731,661, Salcudean, Rohling and Turgay), and many other methods that have been published or patented on the topic. Elastography can be a strain image, shear wave image, shear wave velocity image, Young's modulus image, viscosity image or any other image that depicts a variation in the mechanical properties of tissue. Preferably the elastography methods used will be quantitative, in that they will provide not just a strain image, which provides a relative elasticity measure that depends on the operator, but also quantitative. In some embodiments, the rigidity of the pancreatic tissue is determined by ultrasound elastography. Ultrasound elastography (USE) is a noninvasive method for the determination of tissue stiffness and the measurement value is usually altered by specific pathological or physiological processes of soft tissues. Quantitative ultrasound elastography methods currently include acoustic radiation force impulse (ARFI) and transient elastography (TE) techniques. The term “shear wave elastography” (SWE) refers to the technique of detecting shear-wave velocity (SWV) excited by acoustic radiation forces. Both point shear-wave elastography (pSWE) and two-dimensional shear-wave elastography (2D-SWE) rely on the ARFI technique, which uses focused, short-duration acoustic pulses to deform localized tissue and generate shear waves. Although both pSWE and 2D-SWE use ARFI to generate shear waves, pSWE is often referred to as ARFI elastography in some literature and 2D-SWE is referred to as real-time two-dimensional SWE (RT-2D-SWE).

In some embodiments, the method of the present invention comprises the step of comparing the determined rigidity with a predetermined reference value. Typically, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the expression level of the selected peptide in a group of reference, one can use algorithmic analysis for the statistic treatment of the expression levels determined in samples to be tested, and thus obtain a classification standard having significance for sample classification. The full name of ROC curve is receiver operator characteristic curve, which is also known as receiver operation characteristic curve. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, when the determined rigidity is lower than the predetermined reference value it is concluded that the subject has or is at risk of having a pancreatic cancer.

The method of the present invention is particularly suitable for the early diagnosis of pancreatic cancer. As used herein the term “early diagnosis” refers to an early phase of establishing the existence or degree of pancreatic cancer in the subject, before a symptom or a group of symptoms appears. Thus the method of the present invention is thus particularly suitable for prescribing a treatment therapy suitable for preventing the development of pancreatic cancer. For instance, when it is concluded that the subject is at risk of having a pancreatic cancer, a therapeutically effective amount of a PI3Kα-selective inhibitor. Non-limiting examples of PI3Kα-selective inhibitors are disclosed in Schmidt-Kittler et al., Oncotarget (2010) 1(5):339-348; Wu et al., Med. Chem. Comm. (2012) 3:659-662; Hayakawa et al., Bioorg. Med. Chem. (2007) 15(17): 5837-5844; and PCT Patent Application Nos. WO2013/049581 and WO2012/052745, the contents of which are herein incorporated by reference in their entireties. In particular non-limiting embodiments, the PI3Kα-selective inhibitor is derived from imidazopyridine or 2-aminothiazole compounds. Further non-limiting examples include those described in William A Denny (2013) Phosphoinositide 3-kinase α inhibitors: a patent review, Expert Opinion on Therapeutic Patents, 23:7, 789-799. Further non-limiting examples include BYL719, INK-1114, INK-1117, NVP-BYL719, SRX2523, LY294002, PIK-75, PKI-587, A66, CH5132799 and GDC-0032 (taselisib). One inhibitor suitable for the present invention is the compound 5-(2,6-di-morpholin-4-yl-pyrimidin-4-yl)-4-trifluoromethyl-pyridin-2-ylamine that is described in WO2007/084786, which is hereby incorporated by reference in its entirety hereto. Another inhibitor suitable for the present invention is the compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}- amide) that is described in WO 2010/029082, which is hereby incorporated by reference in its entirety hereto.

The invention will be further illustrated by the following figure and examples. However, these examples and figure should not be interpreted in any way as limiting the scope of the present invention.

### FIGURE

FIG. 1: Longitudinal measure of rigidity in normal, genetically altered pretumoral pancreas and tumors developed in situ reveals a decrease of rigidity before tumor development. Evolution in time of total pancreatic rigidity in kPa. Data represent mean±SEM. n≥4 Unpaired T-test Ctrl vs. KPC at 36-41 w: p=0.0242, *. Two-way unmatched ANOVA test source of variation genotype: p=0.0443, * Control (n=9) or KPC mice (n=5)

### EXAMPLE

### Material & Methods

Animals and Ethical Requirements

The LSL-Kras^{G12D} and LSL-p53^{R172H} knockin (from D Tuveson, Mouse Models of Human Cancers Consortium repository (NCI-Frederick, USA), Pdx1-cre (from DA Melton, Harvard University, Cambridge, Mass., USA) strains were interbred on mixed background (CD1/SV129/C57B16) to obtain compound mutant LSL-Kras^{G12D}; LSL-p53^{R172H}; Pdx1-Cre (named KPC). Littermates not expressing Cre as well as Pdx1-Cre of the same age were used as control. All procedures and animal housing are conformed to the regulatory standards and were approved by the Ethical committee according to European legislation translated to French Law as Décret 2013-118 1 Feb. 2013 (APAFIS 3601-2015121622062840). Genotyping was performed using primers as described in (31) and analyzed with Fragment analyzer instrument (AATI) with dsDNA 910 Reagent kit, 35-1500 bp (AATI). Blood counts (from EDTA tubes Microvette #16.444, SARSTEDT AG & Co) were performed using Yumizen H500 hematology analyzer (HORIBA), calibrated with murine blood.

Imaging Procedure

KPC and control mice (Ctrl) are imaged once a week. The probe SuperLinear™ SL22-7lab (Supersonic Imagine) with preset called “standard” or “optimized” (Supplementary Table 2) was used. After echographic detection of the pancreas, a study box is placed and a US wave is applied focused at different depth, compressing the tissue. The compression of the tissue generate shear-wave perpendicular to US axis, which are measured in live with SuperSonic Aixplorer (France). Colourimetic maps corresponding to the measurement of these waves are immediately available. Elasticity (E) is measured in kiloPascal (kPa), deducted from the velocity of shear waves (E=3ρV_{c}²) in the studied region of interest. Measurement of the shear wave speed results in qualitative and quantitative estimates of apparent tissue elasticity. Tumor volume is calculated as follows: Width²×Length×0.5. Mean detection time of tumour is of 29 weeks; hence this time point was considered as T0 for the Ctrl cohort.

Histology and Immunostaining

Hematoxylin eosin stainings were conducted using standard methods on formalin-fixed, paraffin-embedded tissues. All pancreata were analyzed in blinded fashion. Pancreas were fixed in 10% neutral buffered formalin and embedded in paraffin. For histopathological analysis, pancreata were serially sectioned (4 μm) and every 10 sections stained with hematoxylin and eosin. Histopathological scoring of pancreatic lesions was performed using serial H&E-stained sections (100 μm apart, 2 sections per pancreas). Picro sinus red staining was performed using the manufacturer condition (AbCAM#150681), with minor modifications corresponding to a 30 minute only incubation with the staining reagent.

Immunostainings were conducted using standard methods on formalin-fixed, paraffin-embedded tissues. Antigen retrieval and antibody dilution (p16, F12 clone, Sigma #sc-1661, 1/100, citrate antigen retrieval; F4/80, Cl A3-1 clone, Pierce #MA1-91124, 1/100, Proteinase K antigen retrieval) was carried out as described in table below followed by AEC or DAB incubation prior secondary antibody (ImmPress (MP-7402, Vector) or BA-4001 ( 1/50, Vector)).

Statistics

Experimental data provided at least 3 experimental replicates. Statistical analyses were performed with GraphPad Prism using ANOVA, t-test, or parametric Mann-Whitney tests: * p<0.05, ** p<0.01, *** p<0.001. Non-significant (ns) if p≥0.05. Correlation analysis was performed using Pearson r test.

### Results

Besides mimicking human pathology, KPC mice model the heterogeneous development of pancreatic tumors.

Triple transgenic mice with intrapancreatic mutation of KrasG12D and p53R172H (Pdx1-Cre⁺/− and LSL-Kras^{G12D/+} and LSL-Trp53^{R172H/+} mice, called hereafter KPC) mice were used as a model of locally advanced PDAC that exhibits a typical human-like morphology with abundant desmoplasia, moderate to poor epithelial differentiation and a highly aggressive clinical course (data not shown) (19), and was followed by the newly developed Aixplorer (7-22 MHz probe) (data not shown). This model also progressively develops the preneoplastic lesions prior tumor development albeit at different kinetics in each individual. As previously shown in this model, lethality is gradual, with a medium survival of 213 days, associated with different kinetics of tumor detection (data not shown). An optimized setup allowed the detection of tumors using Aixplorer (data not shown). Tumor detection range was from 19 to 48 weeks. We find that Aixplorer allowed detection at the smallest volume of 0.029 cm³. This imaging method did not allow the detection of preneoplastic lesions based on echographic images. We however found that the loss of striated hyperechogenic areas is an early sign of pancreatic parenchyma modification in both settings, possibly due to defects in normal ducts (data not shown). This observation is dependent on the training of the observer; hence, we next aimed to develop a quantitative method to analyse early modifications in pancreatic parenchyma.

A post-processing quantification is necessary to reproducibly measure elastography in the pancreas.

We then used the elastography module of Aixplorer technology. The elastography measurements were carried out from 17 to 23 weeks old in 3 Ctrl mice. Success rate of imaging both pancreas and kidney in full was not 100%; in most often case, it was due to a swelling of the stomach (data not shown). If not possible, the imaging was repeated the next day. To ensure a repeatability of the measurements, pancreas were imaged at a constant focal zone of −1 cm, with left kidney as a reference organ. The elastograms of pancreas measured in Pascals (Pa) are heterogeneous in time and space, with measured point values ranging from 0.9 to 110 kPa (data not shown). A normal pancreas presents low intensity values (color coding is <30 kPa) with peaks in rigidity (with color coding >30 kPa) (data not shown). These measured elastograms were then post-processed to quantify in vivo rigidity. When we first performed tracing measurements that encompassed the entire organ (total organ area) (data not shown), values were found heterogeneous. Indeed, the presence of red zones in the borderline of the organ are included in the total organ area measurements and increases the standard deviation of the measure, SD being representative of the heterogeneity. Zones which are in contact with stiffer organs such as the stomach also present higher values (yellow colouring observed in pancreas corresponding to values range of 30-35 kPa) (data not shown). To confirm our finding, we compared these data to the rigidity in predefined quantitative boxes of 1 mm in diameter set at distance from the organ margin and blood flows, measure called intra-pancreatic area. The measure of the reference organ acquired in the same image at the same focal zone was also performed (total kidney area, kidney cortex area) (data not shown). The intra-pancreatic measure of the pancreas was taken at distance from other organs and blood vessels on the live image in SWE mode. The average rigidity of the total or intra-pancreatic pancreas and of the total kidney or kidney cortex is 13.24 kPa±0.41, 11.45 kPa±1.41, 13.13 kPa±3.10 and 18.49±1.46 kPa, respectively (based on the average of all images acquired in longitudinal) (data not shown). As expected, intrapancreatic measure was statistically less heterogeneous than the total pancreatic area as measured by the mean SD of these values (data not shown); however, the average rigidity by both methods was not significantly different. Kidney cortex is statistically more rigid than pancreas (total area and intra-pancreatic area). Of note, the total kidney area had a higher heterogeneity value due to the heterogeneous internal areas (medulla) of this organ; kidney being more internal, the full assessment was not possible in all cases leading to the absence of elastogram in its deeper part (data not shown). We confirm that both total pancreatic area and intra-pancreatic area measurements led to measurements of pancreas in accordance to the representative elastograms (data not shown).

Total area, intra-pancreatic area of the pancreas, kidney cortex measurements lead to reproducible and consistent quantification of normal in situ tissular rigidity.

Longitudinal measure of rigidity in Kras and p53 mutated pancreas reveals the inter-individual heterogeneity of tumors developed in situ at detection.

Tumors are known to present an increase of rigidity. We questioned if quantitative measurement of rigidity in pancreas could help to detect in situ developed tumors. We performed tumor detection using B-mode imaging in a cohorte of KPC mice, and longitudinally analyzed body weights and elastography in pancreas with SWE mode (data not shown). SWE mode did not allow to detect tumours earlier; however, the diagnostic of tumor was confirmed by an increase of rigidity. Total tumoral rigidity and maximal intratumoral rigidity point value were not significantly correlated with tumor volume at detection (data not shown). Total tumoral rigidity was correlated with maximal rigidity value (data not shown). Analysis of total tumor rigidity as a function of maximal rigidity value individualized a group of tumours with consistant lower maximal rigidity (data not shown), with values <25 kPa for total tumor rigidity and <60 kPa for maximal rigidity, lower left quadrant.

Experimental in situ PDAC presents a great inter-individual variation, as well as significant heterogeneity within the tumor of each individual. The maximal rigidity area appears as a possible method to discriminate a subtype of experimental pancreatic tumors with lower maximal rigidity.

Intra-tumoral rigidity is heterogeneous and leads to different tumor evolution patterns.

We then followed the elastogram of three tumors for which we had values at detection and at sacrifice (corresponding to the ethical limiting point), one in the lower maximal rigidity group (Black), one borderline (light green) and one with high maximal rigidity values (red). Blood perfusion is known to influence the rigidity values (20). We confirmed using the Doppler imaging mode that pancreatic tumors developed in situ were not perfused (data not shown); in humans, pancreatic tumors are known to be unperfused. We showed that tumors from KPC mice presented heterogeneous intratumoral elastograms both at detection and sacrifice (point values ranging from 0.1 to 108.3 kPa at detection, and from 0.1 to 115.3 kPa at sacrifice). Interestingly, we also observed an inter-tumor heterogeneity with global rigidity ranging from 10.7 kPa (tumor 1) to 26.7 kPa (tumor 3) at detection (data not shown). The global rigidity of the tumors increased between the two time points for the 3 KPC mice (data not shown). Tumoral volume evolution appeared as correlated with global tumoral rigidity evolution (data not shown). The measure of the standard deviation of this global rigidity measurement is a quantitative marker of the intratumoral heterogeneity, by calculating the mean of the point measures in the global area. These values were high for all tumors and increased in time (data not shown). Rigidity evolution also appeared as correlated with the evolution of intratumoral heterogeneity (data not shown). The tumor with the highest global rigidity at detection (Tumor 3-red values) was the tumor which saw its rigidity, its intratumoral rigidity heterogeneity and its volume evolve the less (data not shown).

The maximal rigidity area appears as a possible method to discriminate a type of experimental pancreatic tumors, which evolves slowly.

Longitudinal measure of rigidity in normal, genetically altered pretumoral pancreas and tumors reveals a decrease of rigidity before tumor detection.

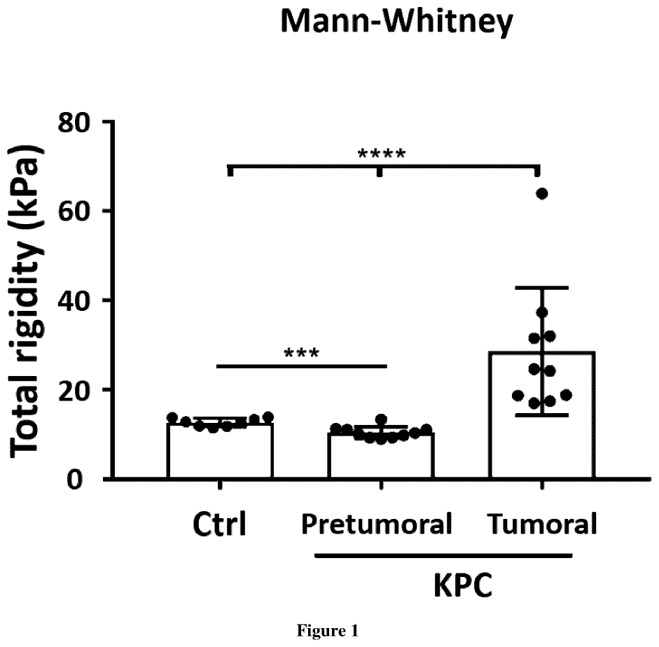

We next questioned if the measure of pancreatic tissular rigidity could detect the microscopic changes in the pancreatic parenchyma which occur before tumor development, possibly helping in an earlier detection of tumors. Indeed, pancreatic oncogenesis develops from preneoplastic lesions, encompassing acino-ductal metaplastic lesions (ADM), pancreatic intraepithelial neoplasia (panIN) or cysts (mucinous cystic neoplasm (MCN), intra-ductal papillary mucinous neoplasm (IPMN). Very frequently (>90% of cases), and at a very early stage (from low grade panIN), the activating mutation of the Kras oncogene is found. These lesions are found in KPC mice. We thus longitudinally analyzed body weights and elastography in pancreas with SWE mode, while tumor detection was performed using B-mode imaging (data not shown). We compared pancreatic elasticity after 23 weeks (data not shown), time at which pro-oncogenic genetic background induced a significant difference in body weights in KPC mice called pretumoral KPC compared to Ctrl mice (data not shown). Pretumoral KPC aged >23 weeks presented a baseline rigidity of total and intra-pancreatic parenchyma with decreased values compared to Ctrl mice (data not shown). In time, KPC pancreas elasticity significantly decreased (FIG. 1). Hence, pro-tumorigenic mutations influence pancreatic elasticity prior to tumor detection. This alteration of pancreatic baseline elasticity preceded the increase in rigidity observed in detected tumors (data not shown).

Ultra fast shear-wave elastography allows the quantification of a pretumoral stage in a reproductive manner and demonstrates in a non invasive manner the increase in mechanical rigidity in tumors.

Markers of senescence are present in the pancreatic pretumoral niche, where increased elasticity was measured.

We then aimed to discover the origin of the baseline modification of elasticity induced by protumoral mutations in the pancreas prior tumor development (data not shown), by analyzing the histology of the pancreas (data not shown). In the pretumoral KPC, we could not detect the presence of preneoplastic lesions. Because we did not analyzed the whole pancreas, we cannot completely refute their presence. Instead, we detected the presence of acinar cells with collapsed morphology, possibly corresponding to senescent cells (data not shown). To confirm the presence of senescent cells only in this condition, the representative cytoplasmic anti-p16 senescent marker was found only in pretumoral condition. This staining is reminiscent to the type of staining previously observed only in preneoplastic lesion by others (21). A significant increase of p16 staining area was detected only in pretumoral KPC as compared to Ctrl (data not shown).

Finally, we searched whether this modification in rigidity of the tissue could be correlated with changes in stainings of F4/80 macrophages, in picro-sirius red positive collagen fibers, or in circulating white blood cell parameters, inflammation being usually associated with senescence in pretumoral condition. For the latter, we analyzed blood counts in the cohorts of mice and plotted granulocyte/lymphocyte (GRA/LYM), neutrophil/lymphocyte (NEU/LYM), and platelet/lymphocyte (PLA/LYM) ratios. We could detect a significant difference in these three parameters in the pretumoral condition; F4/80, picro-sirius red positive stainings, GRA/LYM and NEU/LYM ratios further increased in tumors as compared to Ctrl (data not shown).

Microscopic tissular modifications in the pretumoral tissue where the cancer originates, tissue also described as the pretumoral niche, relate to elastographic measurement modifications. Further modifications of rigidity in tumors is associated with changes in the tissular composition (inflammatory infiltration, collagen deposits). Measure of apparent tissular rigidity by SWE appears as an efficient mode to non-invasively qualify and quantify tissular composition parameters in both pretumoral niche and in locally advanced primary tumors of the pancreas (data not shown).

### Discussion

Advances in therapeutic approaches and molecular subtyping of PDAC (22-26) impose the necessity for individualized early therapeutic diagnostic through an early evaluation of therapy effects in this often rapidly progressing disease. Early therapy-induced effects are probably mirrored in tissue reorganization, changes in number of tumoral and stromal cells (cellularity), cell size or shape, matrix deposition, immune cell recruitment, while measurable changes in tumor volume become apparent at later time points (if any in this pathology). Several novel therapeutic strategies aims at modifying the differentiation of the lesions or the tumoral stroma to modulate their aggressiveness (3). Hence, novel quantitative imaging methodologies based on the physiological changes of the tumors and not based on their volume evolution only will be necessary to assess their early action.

Pancreas is a small organ located deep into the body and strongly affected by aortic pulsation, which complicates the imaging possibilities (5). Increased rigidity is reproducibly observed in tumors as measured by others using other technological approaches (16, 27, 28). Our data favorably argue that ultrafast shear wave elastography is an efficient method to characterize the biophysical properties of pancreas prior tumor development as well as pancreatic tumors in correlation with microscopic changes, rendering this technology an attractive, easy and cheap option to detect pretumoral condition, diagnose tumors and follow therapeutic intervention in basic and preclinical settings.

Surprisingly, in our model, pretumoral niche was associated with a significant baseline lower rigidity in the pancreatic parenchyma. Our data so far favorably argues that elastography detects earlier signs of tumoral development induced by focal oncogenic mutations. These signs appears to be early pancreatic inflammatory parenchyma (as assessed by increased senescence), translated as a decrease in tissue rigidity. In vitro data show that cancer cells present an increased elasticity possibly due to changes in their cytoskeleton properties (29, 30). Because we previously showed that inflammatory conditions in an oncogenic background induces actin cytoskeleton remodelling in exocrine acinar cells and that this intracellular alteration constitutes an early and necessary step in pancreatic lesion formation (31), we speculate that this increase in elasticity found in pancreatic parenchyma is related to a change in actin cytoskeleton polymerisation, indicative of the early stage of cell transformation.

The biomechanical properties of a tissue in terms of elasticity, relatively measured as a rigidity parameter in Pa vary markedly between organs and tissues, and are inherently related to tissue function and content. Increased expression of enzymes modifying extracellular matrix (ECM) by stromal cells results in increased collagen linearization and tissue rigidity in pre-malignant breast tissue (32), rigidity appears to favor tumoral progression (16, 28), while stroma remodeling enzymes such as LOX (Lysyl oxidase) favor metastasis and drug resistance in pancreatic cancer (33). Surgery-induced matrix softening increases the risk of metastasis (34). Further to these findings, our knowledge of the evolution of tumoral mechanical characteristics needs now to be increased to understand the determinants of the impact of tumor solid stress or rigidity. Most studies measuring tumoral rigidity or solid stress describe an increase in these properties which is radially homogeneous within the different layers of the modelled tumors (10); most live mapping of tumor rigidity is made in heterotopic and orthotopic xenografts models (20). These models show that the tissue where the tumoral cells are implanted influences the rigidity measurements (20), further stressing the importance of studying tumor mechanical properties in tumors developed in situ (11). The intrinsic co-development of tumors and of the target organ tumoral niche is likely to influence tumor mechanical properties.

Finally, survival of patients with PDAC does not correlate with total fibrillar collagen content within the tumour, but is significantly linked to the localized increased thickness of collagen fibers directly adjacent to PDAC epithelium (27), leading to a local increased rigidity. Hence, a refined spatial mapping of tumoral and tissular mechanical characteristics could be of importance to determine their clinical relevance. In our model mimicking PDAC heterogeneity, we find that tumors with higher global rigidity evolve less.

In conclusion, as tumors metastasize, some cancer cells within stiffen their microenvironment by increasing the production and cross-linking of collagen. This local rise in rigidity helps the metastatic cells migrate and invade other regions of the body, this occur in coordination with the recruitment of protumoral immune cells. Our data pave the road for the development of novel preclinically and clinically relevant methodologies to assess tumor-environment heterotypic dialog in pancreatic cancer.

### REFERENCES

Throughout this application, various references describe the state of the art to which this invention pertains. The disclosures of these references are hereby incorporated by reference into the present disclosure.

1. Bouvier A M, et al. (2017) Trends in net survival from pancreatic cancer in six European Latin countries: results from the SUDCAN population-based study. European journal of cancer prevention: the official journal of the European Cancer Prevention Organisation 26 Trends in cancer net survival in six European Latin Countries: the SUDCAN study:S63-S69.

2. Bouvier A M, et al. (2017) Focus on an unusual rise in pancreatic cancer incidence in France. International journal of epidemiology 46(6):1764-1772.

3. Neoptolemos J P, et al. (2018) Therapeutic developments in pancreatic cancer: current and future perspectives. Nature reviews. Gastroenterology & hepatology 15(6):333-348.

4. Baer R, Cintas C, Therville N, & Guillermet-Guibert J (2015) Implication of PI3K/Akt pathway in pancreatic cancer: When PI3K isoforms matter? Advances in biological regulation 59:19-35.

5. Lee E S & Lee J M (2014) Imaging diagnosis of pancreatic cancer: a state-of-the-art review. World journal of gastroenterology 20(24):7864-7877.

6. Sigrist R M S, Liau J, Kaffas A E, Chammas M C, & Willmann J K (2017) Ultrasound Elastography: Review of Techniques and Clinical Applications. Theranostics 7(5): 1303-1329.

7. Friedrich-Rust M, Poynard T, & Castera L (2016) Critical comparison of elastography methods to assess chronic liver disease. Nature reviews. Gastroenterology & hepatology 13(7): 402-411.

8. Elyas E, et al. (2017) Correlation of Ultrasound Shear Wave Elastography with Pathological Analysis in a Xenografic Tumour Model. Scientific reports 7(1):165.

9. Herrmann E, et al. (2018) Assessment of biopsy-proven liver fibrosis by two-dimensional shear wave elastography: An individual patient data-based meta-analysis. Hepatology 67(1):260-272.

10. Ambrosi D, Pezzuto S, Riccobelli D, Stylianopoulos T, & Ciarletta P (2017) Solid tumors are poroelastic solids with a chemo-mechanical feedback on growth. Journal Of Elasticity 129(1-2):107-124.

11. Nia H T, et al. (2016) Solid stress and elastic energy as measures of tumour mechanopathology. Nature biomedical engineering 1.

12. El Kaffas A, et al. (2017) Quantitative Three-Dimensional Dynamic Contrast-Enhanced Ultrasound Imaging: First-In-Human Pilot Study in Patients with Liver Metastases. Theranostics 7(15):3745-3758.

13. D'Onofrio M, et al. (2013) Virtual analysis of pancreatic cystic lesion fluid content by ultrasound acoustic radiation force impulse quantification. Journal of ultrasound in medicine: official journal of the American Institute of Ultrasound in Medicine 32(4):647-651.

14. Kuwahara T, et al. (2018) Usefulness of shear wave elastography as a quantitative diagnosis of chronic pancreatitis. Journal of gastroenterology and hepatology 33(3):756-761.

15. Pfahler M H C, et al. (2018) Point shear wave elastography of the pancreas in patients with cystic fibrosis: a comparison with healthy controls. Abdominal radiology.

16. Marangon I, et al. (2017) Tumor Stiffening, a Key Determinant of Tumor Progression, is Reversed by Nanomaterial-Induced Photothermal Therapy. Theranostics 7(2):329-343.

17. D'Onofrio M, et al. (2014) Elastography of the pancreas. European journal of radiology 83(3):415-419.

18. Fernandez-Sanchez M E, et al. (2015) Mechanical induction of the tumorigenic beta-catenin pathway by tumour growth pressure. Nature 523(7558):92-95.

19. Hingorani S R, et al. (2005) Trp53R172H and KrasG12D cooperate to promote chromosomal instability and widely metastatic pancreatic ductal adenocarcinoma in mice. Cancer cell 7(5):469-483.

20. Riegler J, et al. (2018) Tumor Elastography and Its Association with Collagen and the Tumor Microenvironment. Clinical cancer research: an official journal of the American Association for Cancer Research 24(18):4455-4467.

21. Guerra C, et al. (2007) Chronic pancreatitis is essential for induction of pancreatic ductal adenocarcinoma by K-Ras oncogenes in adult mice. Cancer cell 11(3):291-302.

22. Bailey P, et al. (2016) Genomic analyses identify molecular subtypes of pancreatic cancer. Nature 531(7592):47-52.

23. Collisson E A, et al. (2011) Subtypes of pancreatic ductal adenocarcinoma and their differing responses to therapy. Nature medicine 17(4):500-503.

24. Moffitt R A, et al. (2015) Virtual microdissection identifies distinct tumor- and stroma-specific subtypes of pancreatic ductal adenocarcinoma. Nature genetics 47(10):1168-1178.

25. Sivakumar S, de Santiago I, Chlon L, & Markowetz F (2017) Master Regulators of Oncogenic KRAS Response in Pancreatic Cancer: An Integrative Network Biology Analysis. PLoS medicine 14(1):e1002223.

26. Waddell N, et al. (2015) Whole genomes redefine the mutational landscape of pancreatic cancer. Nature 518(7540):495-501.

27. Laklai H, et al. (2016) Genotype tunes pancreatic ductal adenocarcinoma tissue tension to induce matricellular fibrosis and tumor progression. Nature medicine 22(5):497-505.

28. Levental KR, et al. (2009) Matrix crosslinking forces tumor progression by enhancing integrin signaling. Cell 139(5):891-906.

29. Suresh S (2007) Biomechanics and biophysics of cancer cells. Acta biomaterialia 3(4):413-438.

30. Pegoraro A F, Janmey P, & Weitz D A (2017) Mechanical Properties of the Cytoskeleton and Cells. Cold Spring Harbor perspectives in biology 9(11).

31. Baer R, et al. (2014) Pancreatic cell plasticity and cancer initiation induced by oncogenic Kras is completely dependent on wild-type PI 3-kinase p110alpha. Genes & development 28(23):2621-2635.

32. Pickup M W, Mouw J K, & Weaver V M (2014) The extracellular matrix modulates the hallmarks of cancer. EMBO reports 15(12):1243-1253.

33. Miller B W, et al. (2015) Targeting the LOX/hypoxia axis reverses many of the features that make pancreatic cancer deadly: inhibition of LOX abrogates metastasis and enhances drug efficacy. EMBO molecular medicine 7(8):1063-1076.

34. Rachman-Tzemah C, et al. (2017) Blocking Surgically Induced Lysyl Oxidase Activity Reduces the Risk of Lung Metastases. Cell reports 19(4):774-784.

1. A method of determining whether a subject has or is at risk of having a pancreatic cancer comprising determining the rigidity of the pancreatic tissue of the subject wherein the determined rigidity indicates whether the subject has or is at risk of having a pancreatic cancer.

2. The method of claim 1 wherein the pancreatic cancer is pancreatic ductal adenocarcinoma.

3. The method of claim 1 wherein the subject exhibits one or more risk factors for pancreatic cancer.

4. The method of claim 1 wherein the subject suffers from a pancreatitis.

5. The method of claim 1 wherein the rigidity of the pancreatic tissue is assessed by elastography.

6. The method of claim 1 wherein the rigidity of the pancreatic tissue is assessed by ultrasound elastography.

7. The method of claim 1 wherein the rigidity of the pancreatic tissues is assessed by shear wave elastography.

8. The method of claim 1 which comprises the step of comparing the determined rigidity with a predetermined reference value.

9. The method of claim 8 wherein when the determined rigidity is lower than the predetermined reference value it is concluded that the subject has or is at risk of having a pancreatic cancer.

10. (canceled)

11. A method of diagnosing and treating pancreatic cancer in a subject in need thereof, comprising determining the rigidity of the pancreatic tissue of the subject, and
administering to the subject a therapeutically effective amount of a pancreatic cancer treatment when the determined rigidity is lower than a predetermined reference value.

12. The method of claim 11, wherein the pancreatic cancer treatment is a PI3Kα-selective inhibitor.
